(19) United States
(12) Patent Application Publication
(10) Pub. No.: US 2011/0008830 A1
(43) Pub. Date: Jan. 13, 2011

(54) PROCESS FOR PRODUCING BIO-GEL AND A BIO-GEL

(75) Inventors: Andrew Desbarats, Aurora (CA); Vince Yacyshyn, Aurora (CA)

Correspondence Address: Manelii Denison & Selter PLLC, 2000 M Street, 7th Floor, Washington DC, DC, 20036, US

(73) Assignee: SCIENTEK LLC, Phillpsburg, KS (US)

(21) Appl. No.: 12/922,731

(22) PCT Filed: Mar. 13, 2009
(86) PCT No.: PCT/IB2009/005446

§ 371 Date: Sep. 15, 2010

Related U.S. Application Data
(60) Provisional application No. 61/037,075, filed on Mar. 17, 2008.

(51) Int. Cl. C12P 21/00; C07K 14/00

(52) U.S. Cl. 435/68.1; 530/350

(57) ABSTRACT

Provided is a process for producing an enzyme-containing bio-gel from an enzyme solution, particularly a commercial α-amylase solution. When allowed to stand in a vessel, enzyme from the enzyme solution collects on the inside surface of the vessel, thus forming a bio-gel. The bio-gel is recovered by removing the enzyme solution, then collecting, and optionally drying, the material deposited on the inner surface of the vessel. A bio-gel produced by this process is also provided.

This application claims priority to U.S. Patent App'n Ser. No. 61/037,075, filed 17 Mar. 2008, the complete disclosure of which is incorporated herein by reference.

### FIELD OF THE INVENTIONS

The inventions relate to processes for producing a bio-gel from an enzyme solution and a dried bio-gel formed by the processes.

### BACKGROUND OF THE INVENTION

Commercial enzyme solutions are now well-known. The commercial enzyme solutions are used to form products, such as alcohols and syrups from starch. The commercial enzyme solutions are expensive and, thus, there is a need for further useful products derived from the commercial enzyme solutions to recover costs.

### SUMMARY OF THE INVENTIONS

An embodiment of the invention provides a method of making a bio-gel comprising storing an enzyme solution in a reaction vessel for a time period and under conditions such that a bio-gel is formed on an inside surface of the reaction vessel and as a precipitate suspended in solution. Once the bio-gel is formed, the enzyme solution can be removed from the reaction vessel and used in any known manner to produce products. The bio-gel is also removed from the reaction vessel.

Another embodiment of the invention provides a dried bio-gel formed by storing an enzyme solution in a reaction vessel for a time period and under conditions such that a bio-gel is formed on an inside surface of the vessel and as a precipitate suspended in solution, removing the bio-gel from the reaction vessel, and drying the bio-gel.

Still another embodiment of the invention provides a method of making a bio-gel comprising storing an enzyme solution in a reaction vessel for a time period and under conditions such that a bio-gel is formed in an inside surface of the reaction vessel and as a precipitate suspended in solution. Immobilized on this bio-gel is a portion of the enzyme from the enzyme solution that was used to make the bio-gel. The immobilized enzyme retains its activity.

### BRIEF DESCRIPTION OF THE DRAWING

FIG. 1 is a side view of a reaction vessel having a bio-gel formed on at least a portion of an inside surface of the reaction vessel.

### DETAILED DESCRIPTION OF THE INVENTIONS

The inventions will now be explained with reference to the attached FIGURE without being limited thereto.

Commercial enzyme solutions are now well known, such as those sold by Novozymes. The bio-gel can be formed according to the present inventions using any desired commercial enzyme solution that is formulated with a polyol, alcohol ethoxylate or other surfactant. Preferably, the enzyme comprises at least one group 3 hydrolase. A most preferred enzyme is amylase.

The biogel is formed prior to use of the enzyme in a bioreactor. Preferably, the biogel is formed in the substantial absence of substrates, such as cellulose materials and starches.

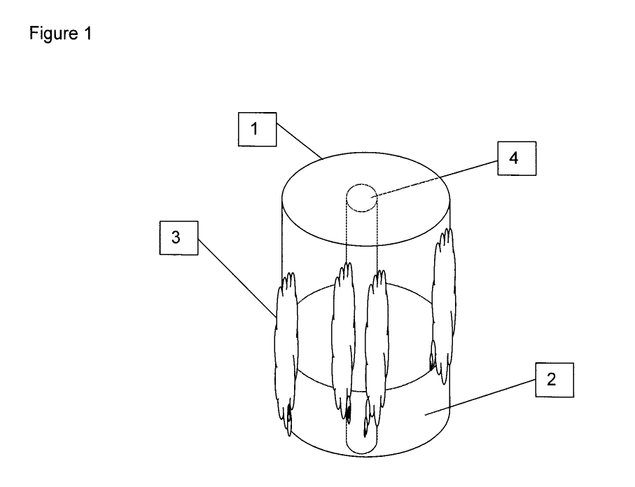

FIG. 1 shows a reaction vessel 1 containing enzyme solution 2. Based on the disclosure provided herein, one skilled in the art will now be able to form a bio-gel 3 from any desired commercial enzyme solution that is formulated with a polyol, alcohol ethoxylate or other surfactant 2, without undue experimentation. The bio-gel 3 can be formed by storing the enzyme solution 2 in the reaction vessel 1 for a time period and under conditions such that a bio-gel 3 is deposited on the inside surfaces of the reaction vessel 1 as well as on the surface of any objects 4 within the reaction vessel 1 and as a precipitate suspended in the enzyme solution. The reaction conditions should be such that the viability of the enzyme is not degraded or destroyed.

Typical commercial enzyme solutions contain a high concentration of dissolved salts. If such a commercial enzyme solution is used, a condition for forming the bio-gel can be reducing the salt concentration of the enzyme solution, such as by adding water and/or by adding a buffer solution. For example, when using commercially available alpha amylase solutions, we have found that the salt concentration can be reduced by mixing the enzyme solution with between 3 parts buffer to 1 part enzyme and 100 parts buffer to 1 part enzyme, preferably between 3 parts buffer to 1 part enzyme and 15 parts buffer to 1 part enzyme, to reduce the salt concentration to a level whereby a bio-gel is formed during storage. The buffer can be any conventional buffer that is suitable for use with enzymes, which are now well known. Preferred buffers comprise at least one of phosphate buffer, citrate buffer, succinate buffer or acetate buffer.

Similarly, commercial enzymes are often prepared in solutions containing alcohol ethoxylates and/or polyol compounds. In these commercial enzyme preparations a bio-gel can be formed by diluting with water or buffer at a ratio of 1 part enzyme 5 parts buffer up to 1 part enzyme 100 parts buffer.

FTIR analysis of a formed bio-gel shows a small but distinct peak at 1740 cm⁻¹. This is normally assigned to a carbonyl group and indicates presence of an ester or lactone. This peak was not apparent in the commercial enzyme from which the bio-gel was derived.

In addition, a broad peak at 2900 cm⁻¹ signifies an increased C—H stretch mode, relative to the commercial enzyme from which the bio-gel was derived.

Finally, a significant decrease in the broad peak at 3350 cm⁻¹ indicates a decrease in the presence of O—H stretching relative to the commercial enzyme from which the bio-gel was derived.

An interesting property of the bio-gel is that there is enzyme activity associated with it. Based on enzyme activity assays where the commercial enzyme is amylase and the substrate is corn mash at 80° C., the enzyme activity of the amylase immobilized within the biogel is approximately half that of the activity of the commercial enzyme. Activity was measured as the increase in % sugar in the mash over time as observed on a refractometer. It is an important aspect of the present invention that by reducing the salt and polyol concentration of a commercial enzyme preparation, an immobilization media and an immobilized enzyme on that media can be created simultaneously.

FTIR analysis confirms the presence of protein in an exemplary bio-gel. Both the commercial enzyme preparation and the bio-gel show a significant peak at 1540⁻¹-1560 cm⁻¹. This range is normally assigned to an amide II vibration which is a combination of largely N—H bending and C—N stretching vibrations [R′CONHR″].

A thermogravimetric comparison of a dried sample of the bio-gel and a similarly dried sample of the commercial enzyme preparation from which it was derived indicates that the dried bio-gel displayed a significant weight loss at 710° C. and under oxygen gas relative to the dried commercial enzyme preparation. In contrast, the commercial enzyme preparation, from which the biogel was derived, showed the majority of weight loss at 221° C. under nitrogen gas. For the purposes of this thermogravimetric analysis, nitrogen gas is replaced with oxygen gas above 700° C.

The time period for deposition of the bio-gel from solution can be adjusted as desired for the particular application. We have found that for a salt-reduced, commercial alpha amylase solution, the storage should be a minimum of about 0.5 hours. While not being bound by any maximum time period, we have found that most of the bio-gel is deposited from a salt-reduced, alpha amylase solution in about 96 hours. Based on the disclosure provided herein, one skilled in the art will be able to adjust the conditions to increase or decrease the deposition rate of the bio-gel as desired. We have found that in the case of alpha amylase, the bio-gel formed more quickly as the concentration of a phosphate buffer was increased. On the low end, bio-gel formed over a period 20 days when a phosphate buffer strength of 0.01 mol/L was utilized and, on the high end, the bio-gel formed in as little as 3 days at a phosphate buffer concentration of 0.7 mol/L.

The rate of formation of the bio-gel can be delayed by including a polyol an alcohol ethoxylate or another surfactant in the buffer solution. For example, bio-gel formation is delayed by approximately 48 hours when a 5% (v/v) solution of Polysorbate-20 (Tween-20) in water is used.

The temperature should be selected such that the viability of the enzyme is not degraded or destroyed. Ambient temperatures are preferred, but any temperature desired can be used.

The process of forming bio-gel can be repeated as desired to successively build up layers of bio-gel on the inside surface of the vessel, or any other desired surface. As shown in the Example, if desired, a continuous stream of enzyme solution can be run through the reaction vessel to continuously form bio-gel.

The soluble enzyme solution that was used to make the bio-gel can be removed from the reaction vessel after formation of the bio-gel and then used in any conventional manner, such as to hydrolyze starch and/or maltodextrin to form products such as alcohols and syrups. In the case of other Group 3 hydrolase enzymes, such as cellulase and xylanase, the soluble enzyme can be used to hydrolyze cellulose cellobiose and xylose. Preferred products are high fructose corn syrup and ethanol. Alternatively, the enzyme solution that was used to make the bio-gel can be used as a liquid medium for bacterial or fungal growth.

In the case of salt reduced alpha-amylase, we found that the resulting enzyme solution had a specific gravity of between 1 and 1.05 g/mL and a conductivity of between 1 and 10 mS/cm after formation of the bio-gel. Preferably, if a concentrated enzyme solution is used as the starting material, the enzyme solution is diluted to a specific gravity of about 1 g/ml.

Once the bio-gel is formed, we have found that in the case of alpha amylase, the bio-gel often liquefied if it was not dried. Thus, preferably, the bio-gel is dried after formation to provide longer term stability.

The bio-gel and the liquid enzyme-buffer mixture are excellent media for the growth of fungus and bacteria.

We have found that surprisingly the formation of the bio-gel does not negatively affect the activity of the enzyme solution for use in a bioreactor. Thus, conventional alcohol and syrup production facilities utilizing enzyme solutions can easily be modified based on the novel teachings provided herein to include a reaction vessel for producing bio-gel, without negatively impacting production of the alcohol and syrup. Preferably, the enzyme solution removed from the reaction vessel after formation of the bio-gel is used within 24 hours of removal from the reaction vessel in an alcohol or syrup production facility.

### EXAMPLE

A non-limiting example of a modified commercial fuel ethanol production process containing a reaction vessel for producing bio-gel is provided.

In dry-mill fuel ethanol plants, commercial alpha amylase enzyme solution is often added at two locations: 1) the slurry tank; and 2) the liquefaction tank. In this particular example, commercial alpha amylase solution destined for the liquefaction tank was diverted to a mixing tank where it was mixed for 30 minutes with 10 parts 0.02M sodium phosphate buffer at pH 6, at ambient temperature, to provide a salt-reduced, buffer-enzyme mixture. The buffer-enzyme mixture was then pumped to a reaction vessel where it was subsequently slowly pumped to the liquefaction system in the fuel ethanol production process. The reaction vessel was continuously replenished with fresh buffer-enzyme mixture. While in the reaction vessel, and before being sent to the liquefaction system, the buffer-enzyme mixture produced bio-gel on the inside surface of the reaction vessel and as a precipitate suspended in solution. The bio-gel was scraped off the inside surface of the reaction vessel and then dried. The bio-gel was also filtered from the soluble enzyme solution and then dried. The production of the bio-gel did not negatively impact the ethanol production process. In this manner, the dried bio-gel can be produced as a by-product to provide a source of additional revenue in industries that hydrolyze starch with enzymes.

An alternate use for the bio-gel is to provide an immobilized enzyme system that can be used either in the plant to hydrolyze starch (in the case of amylase), cellulose (in the case of cellulase) and xylose (in the case of xylanase), or sold to other industries that use immobilized enzymes.

While the claimed invention has been described in detail and with reference to specific embodiments thereof, it will be apparent to one of ordinary skill in the art that various changes and modifications can be made to the claimed invention without departing from the spirit and scope thereof.

We claim:

1. A method of producing a bio-gel comprising:
storing an enzyme solution in a reaction vessel under conditions and for a time period to form a bio-gel on at least a portion of an inside surface of the reaction vessel, prior to use of the enzyme solution in a bioreactor;
removing the enzyme solution from the reaction vessel; and
removing the bio-gel from the reaction vessel.

2. The method according to claim 1, wherein the enzyme solution comprises at least one group 3 hydrolase.

3. The method according to claim 2, further comprising reducing the salt concentration of the enzyme solution.

4. The method according to claim 3, wherein the salt concentration is reduced by mixing the enzyme solution with between 3 parts buffer to 1 part enzyme and 100 parts buffer to 1 part enzyme.

5. The method according to claim 3, wherein the salt concentration is reduced by mixing the enzyme solution with between 3 parts buffer to 1 part enzyme and 15 parts buffer to 1 part enzyme.

6. The method according to claim 4, wherein the buffer comprises at least one of a phosphate buffer, citrate buffer, succinate buffer or acetate buffer.

7. The method according to claim 3, wherein the salt concentration is reduced by mixing the enzyme solution with an aqueous solution.

8. The method according to claim 7, wherein the aqueous solution comprises water.

9. The method according to claim 1, wherein the enzyme solution is stored in the reaction vessel for a minimum of about 0.5 hours.

10. The method according to claim 9, wherein the enzyme solution is stored in the reaction vessel for a maximum of about 96 hours.

11. The method according to claim 1, further comprising storing and removing enzyme solutions from the reaction vessel to build up layers of the bio-gel on the inside surface of the reaction vessel.

12. The method according to claim 1, further comprising continuously running the enzyme solution through the reaction vessel to continuously produce the bio-gel.

13. The method according to claim 1, further comprising drying the bio-gel.

14. The method according to claim 1, further comprising reacting the enzyme solution removed from the reaction vessel in a bioreactor with at least one of starch or maltodextrin to form a product.

15. The method according to claim 14, wherein the product comprises a corn-syrup.

16. The method according to claim 14, wherein the product comprises an alcohol.

17. The method according to claim 1, wherein the bio-gel is coated on a surface of an object contained within the reaction vessel.

18. The method according to claim 1, further comprising using the enzyme solution removed from the reaction vessel as a medium for growing fungus or bacteria.

19. The method according to claim 1, further comprising using the bio-gel removed from the reaction vessel as a medium for growing fungus or bacteria.

20. The method according to claim 1, wherein the enzyme is stored in the reactor in the substantial absence of starch or cellulose materials.

21. The method according to claim 1 wherein a portion of the enzyme in the commercial enzyme solution is immobilized on the bio-gel.

22. The method according to claim 21, wherein the immobilized enzyme retains it's intrinsic activity.

23. The method according to claim 22 wherein the immobilized enzyme-containing bio-gel is removed from the reaction vessel and dried.

24. The method according to claim 22 wherein the immobilized enzyme-containing bio-gel is removed from the reaction vessel and stored such that moisture in the bio-gel is retained.

25. The method according to claim 1 wherein the bio-gel has a distinct FTIR peak in the range of 1730-1750 cm⁻¹

26. The method according to claim 1 wherein the bio-gel has a broad FTIR peak in the range of 2900 cm⁻¹

27. The method according to claim 1 wherein the bio-gel has a diminished peak in the 3300 cm⁻¹ range relative to the commercial enzyme preparation from which it was prepared.

28. The method according to claim 1 wherein the bio-gel sample displays a majority of it's weight loss at 710° C. under oxygen gas.

29. The method according to claim 1 wherein the bio-gel is removed from the reaction vessel by filtering the enzyme solution.

30. A bio-gel formed from storing an enzyme solution in a reaction vessel substantially free-of starch or cellulose materials under conditions and for a time period to form the bio-gel on at least a portion of an inside surface of the reaction vessel and removing the bio-gel from the reaction vessel.

31. The bio-gel according to claim 30, wherein the bio-gel has been dried.
